(19) United States
(12) Patent Application Publication
(10) Pub. No.: US 2022/0018832 A1
(43) Pub. Date: Jan. 20, 2022

(54) Materials and Methods for Glycan Profiling

(71) Applicants: GlycoTechnica Co. Ltd., Yokoma-shi (JP); National Center for Child Health and Development, Tokyo (JP)

(72) Inventors: Akihiro Umezawa, Tokyo (JP); Koji Okamura, Tokyo (JP); Mayu Shibata, Tokyo (JP); Masao Yamada, Yokohama (JP)

(21) Appl. No.: 16/933,835

(22) Filed: Jul. 20, 2020

(51) Int. Cl. G01N 33/53; G01N 33/543; G01N 21/64; G16H 50/20

(52) U.S. Cl. CPC G01N 33/5308; G01N 33/54306; G01N 21/6428; G01N 2570/00; G01N 2440/38; G01N 2021/6439; G01N 2800/52; G16H 50/20

(57) ABSTRACT

The invention aims to provide a non-destructive method of testing and analysing, amongst other things, cell types such as pluripotent cells, including differentiated cells and MSCs. The invention also aims to provide a non-destructive method to test and analyse the cell conditions, such as aging, activity, multipotency, differentiated directivity and effectiveness. Possibility of analysing cell types or cell conditions without destroying the cells by analyzing glycan profile, which is acquired from glycoconjugates secreted from cells in culture mediums, using microarrays with glycan binding protein, on the surface was discovered.

### FIELD OF THE INVENTION

This invention is related to testing device and analysis method which examine the state of cells in a non-destructive manner. This invention's testing and analysis are related to classification and analysis of cells such as pluripotent cells including differentiated cells and stem cells. Furthermore, the states of cells which are to be tested and analysed by this invention include aging and activity, multipotency, differentiated directivity and effectiveness.

### BACKGROUND OF THE INVENTION

Various testing devices and analysis methods are known with regards to testing the state of cells in a non-destructive manner.

Patent literature 1 describes sample chip analysis device and a method with regards to analysing gene expression aspect of cells and biological tissues, and antigen-antibody reaction. The device and the method detect the emission from fluorescent substance labeled and bound in test samples, accurately and with high precision, optimising analysis process of test samples without being influenced by excitation light from fluorescent substance and ambient light.

Patent literature 2 proposes a method to analyse interaction between protein and glycan which can lead to practical application of lectin arrays. Base plate with proteins such as: lectins; enzyme proteins which possesses sugar binding domain; cytokines which possess affinity to glycans; or antibodies which indicate interaction with glycans, immobilized on the surface, is made to come into contact with specimen glycans or specimen complex carbohydrates which have been fluorescent labeled. This base plate is left unwashed and is effected with exciting light (evanescent wave). Through this process, interaction between protein and glycan is analyzed by measuring the strength of fluorescence of the excitation.

Patent literature 3 describes a device which analyzes glycans or complex carbohydrates by a method of measuring the strength of fluorescence which is generated through the following method for each placement location of the glycan binding proteins mentioned below. The Process involves introducing light to a base plate which consists of optical guiding material with glycan binding proteins, belonging to IgM class such as glycan recognizing antibodies and lectins, arranged in multiple kinds fixing and to the side edge of this base plate to excite fluorescent label by generating evanescent wave on the surface of the base panel.

The following are stipulated as minimum requirements for quality control for evaluation condition of mesenchymal stromal cells (hereinafter referred to as ‘MSCs’) (non-patent literature 1):

1. They can be bonded to plastic and be cultivable.

2. Regarding cell surface markers' expression, CD73, CD90, CD105 are positive, CD45, CD34, CD14 or CD11b, CD79a or CD19, HLA-DR are negative.

3. Having the ability to be differentiated into osteoblasts, adipocytes and chondrocytes.

However, testing differentiation potency requires much time. Furthermore, testing cell surface markers can be conducted with flow cytometer so it is relatively simple. However, specificity of these markers cannot be said to be high and it is pointed out that there is a limit to cells which can be identified. Specifically, distinguishing between MSCs and fibroblast is extremely difficult (non-patent literature 2).

One of the evaluation methods recommended by the International Society for Cellular Therapy for the formulation of MSC-like cellular products includes evaluation of the regulation of immune function, and stimulation of cytokines such as ITFNγ and TNFα and gene expression profiling are recommended (non-patent literature 3).

This is because with regards to MSCs' anti-inflammatory action, it is considered to be possible to predict, to a degree, which factors can be induced to what extent as a result of cytokine stimulation, and it is thought that in the field of regenerative medical products using MSCs, it can lead to its effectiveness (non-patent literature 4).

Regarding the evaluation of regulation of immune function, increased cost, because of the necessity for cytokine stimulation and for further cultivation of cells which means more time is required for testing, is cited as a problem. Nucleic acid extraction is necessary for such an evaluation and this means that destruction of cells is involved. A method for conducting glycan profiling by collecting exosome from culture supernatant is known (non-patent literature 5) but collection of exosome requires concentration operation using ultracentrifugation and therefore cannot be said to be highly convenient.

Therefore regarding quality control protocols for the development of regenerative medical products using MSCs, there is a scope for improvement in terms of time and cost etc.

### PATENT LITERATURE

Patent application JP 2003-172701 official journal
International Publication No. WO2005/064333.
International Publication No. WO2006/137311

### Non Patent Literature

M. Dominici, K. Le Blanc, I. Mueller, I. Slaper-Cortenbach, F. Marini, D. Krause, R. Deans, A. Keating, Dj. Prockop, E. Horwitz, Minimal criteria for defining multipotent mesenchymal stromal cells. The International Society for Cellular Therapy position statement, Cytotherapy, 2006; 8(4), pp 315-317.
E. Alt, Y. Yan, S. Gehmert, Y H Song, A. Altman, S Gehmert, D. Vykoukai, X. Bai, Fibroblasts share mesenchymal phenotypes with stem cells, but lack their differentiation and colony-forming potential, Biol. Cell., 2011 April; 103(4) pp. 198-208 Cytotherapy, 2016 February; 18(2):151-159. doi:10.1016/j/jcyt.2015.11.008.
Galipeau et al., 2016 Cytotherapy; & Chinnadurai et al., 2018 Cell Reports.
A. Shimoda et al., BBRC 491 (2017), pp. 701-707

### SUMMARY OF THE INVENTION

At its most general, the present invention provides a method of testing and analysing cells based on their glycan profile where the method avoids the destruction of the cells. Glycans in glycoproteins and glycolipids play many essential roles in cells, such as protein folding, stability, cell adhesion, regulation of cell differentiation and proliferation. The method of the invention allows cells to be categorised according to age, activity, multipotency, differentiated directivity and effectiveness based on a determined glycan profile.

The present invention particularly provides a method of testing and analysing cell types such as pluripotent cells including differentiated cells. Of particular interest are mesenchymal stromal cells (MSC)s and their derived cells, osteoblasts, adipocytes and chondrocytes. The glycan structures vary depending on the cell type and change during events such as differentiation and oncogenesis. Accordingly, the analysis of glycan profile of cells provides useful information relating to the cellular status and condition. An improved method, such as described herein, will provide standardization and quality control for clinical uses and regenerative medical products.

As a result of earnest investigation, the inventors discovered that it is possible to analyse cell types or cell status and condition without destroying the cells by analyzing glycan profile which is obtained from glycans secreted from cells, using microarrays with glycan binding proteins immobilized on the surface. The invention was completed based on such knowledge. This provides major improvements over the methods in the art which require procedures such as centrifugation and ultracentrifugation of the cells in order to obtain the glycanconjugates (e.g. glycoproteins) of interest which leads to the destruction of the cells and/or the methods require washing steps which risks breaking the weak interactions between the glycans of interest and the glycan binding proteins on the solid support.

In a first aspect of the invention there is provided a method for determining the glycan profile of a cell of interest, said method comprising the steps of
(a) culturing the cell of interest in a culture medium for a period of time;
(b) following said period of time, collecting a sample of supernatant from the culture medium, wherein the supernatant is cell-free;
(c) mixing the cell-free supernatant with a fluorescent labelling agent to create a labelled sample solution, wherein the fluorescent labelling agent is capable of fluorescently labelling a glycoconjugate;
(d) contacting the labelled sample solution with a plurality of glycan binding proteins under conditions suitable to allow binding of a fluorescently labelled glycoconjugate to bind to at least one of said plurality of glycan binding proteins thereby forming a fluorescent labelled glycoconjugate-glycan binding protein complex, wherein said plurality of glycan binding proteins are immobilized on a solid support;
(e) applying an excitation light and measuring the level of intensity of excited fluorescence generated from the fluorescent labelled glycoconjugate-glycan binding protein complex;
(f) determining a glycan profile of the cell of interest based on the intensity of excited fluorescence generated.

In an embodiment, the glycoconjugate is selected from a glycoprotein, proteoglycan or glycolipid. In a preferred embodiment, the glycoconjugate is a glycoprotein.

In a further preferred embodiment, the cell of interest is a human cell, preferably selected from a stem cell, a pluripotent stem cell, or a differentiated cell. In a particularly preferred embodiment, the cell of interest is a human mesenchymal stromal cell.

In a further embodiment of the invention, the culture medium is preferably a serum-free culture medium.

The period of time for culturing the cells of interest is preferably greater that 1 hour, greater than 5 hours, greater than 12 hours, greater than 24 hours, greater than 48 hours, greater than 3 days, greater than 4 days or greater than 5 days. In certain embodiments, the period of time may be between 1 hour and 24 hours, between 12 hours and 24 hours, between 24 hours and 48 hours, between 2 days and 4 days, or between 2 days and 5 days. In an embodiment of the invention, the fluorescent labelling agent is a substance that has the ability to fluorescently label a glycoconjugate, for example a glycoprotein. For example, the fluorescent labelling agent is selected from the group consisting of 2-aminopyridine, Cy3, Cy3.5, Cy5 and tetramethyl rhodamine. In a particularly preferred embodiment, the fluorescent labelling agent is Cy3.

The glycan binding protein comprise proteins or peptide capable of binding to sugar chains. These include lectins, enzymes with a sugar-binding domain, cytokines having binding affinity for sugar chain molecules or antibody binding domains capable of binding to sugar chains. In a preferred embodiment, the glycan binding protein is a lectin. Lectin microarray chips are available in the art, e.g. LecChip™; GlycoTechnica.

The fluorescence generated by the excitation light is measured for fluorescent intensity. The excitation light is may be generated from any source such as a light source spliced from white light, a laser light and more preferably an evanescent wave.

In a further preferred embodiment, the method uses an evanescent wave excitation fluorescence scanner. In a still further preferred embodiment, the method uses an evanescent-filed fluorescent-assisted (EFF) lectin array system for analyzing glycan profiles. These systems allow the evanescent wave to excite a fluorescent field in the vicinity of the solid support (e.g. glass slide) within a distance of less than 200 nm allowing direct detection and increased sensitivity of the glycan-lectin interactions. An example of such a system is GlycoStation® Tools Pro Suite 1.5 (GlycoTechnica). Examples of other evanescent wave excitation fluorescence scanner include GlycoStation Reader and GlycoLite manufactured by GlycoTechnica Ltd.

This invention as described herein provides a non-destructive method to test and analyse cell types such as pluripotent cells, including differentiated cells and MSCs. The invention will also provide a non-destructive method to test and analyse cell conditions, including aging, activity, multipotency, differentiated directivity and effectiveness. Since the method of testing and analysis in the invention do not require any pretreatment, it is possible to reduce time and cost in terms of quality control in the development of regenerative medical products which makes use of cells. In particular, the method of the invention does not require any ultracentrifugation step which destroys the cells. The inventors have found that the culture medium supernatant comprises secreted glycans (for example, in the form of glycoproteins) that allow a glycan profile to be determined for the cells of interest. The supernatant can be collected without the need for time consuming or destructive processes that break down the cells.

The non-destructive methods for obtaining a glycan profile of a cell of interest as provided herein may be used to diagnose, assess or provide prognosis of a disease in an individual. For example, in an embodiment of the invention, the glycan profile obtained for the cell of interest could be compared with known, reference glycan profiles for that cell type. If differences are determined between glycan profile of the cell of interest and the reference glycan profile for that cell type, this may be indicative of a change in status of the cell obtained from the subject. For example, it may be a change in status from a normal cell to a tumor cell; it may indicate a viral infection; it may indicate a change in differentiation status.

Accordingly, in a further aspect of the invention, there is provided a method of determining a change in cell status, e.g. for the purpose of diagnosing, assessing, and/or prognosing, a disease in a subject, the method comprising:
(i) comparing a glycan profile for a cell of interest obtained from the subject with a reference glycan profile for the same cell type, wherein said glycan profile for the cell of interest has been determined by a method according to any one of claims 1 to 13; and
(ii) determining any differences in the glycan profile between the cell of interest obtained from the subject and that of the reference cell, wherein any difference between the glycan profile of the cell of interest and that of the reference cell is indicates a change in states of the cell and may indicate the presence or degree of disease in a subject.

In one embodiment, the disease is selected from the group consisting of cancer, virus infection, bacterial infection and autoimmune disease. Any type of cell can be a cell of interest. For instance, the cell of interest could be a suspected cancer cell, and the obtained glycan profile is compared to normal cells. Any differences in the glycan profile may predict disease progression. As another example, in the case of cells infected by viruses versus normal cells, the glycan profile may provide information, allowing identification of infected cells or to predict the infection status and the disease progression.

In one embodiment of the invention, the reference glycan profiles are stored in a memory unit of a classification system.

In a further aspect of the invention, there is provided a glycan profile classification system comprising a glycan profile classification apparatus and an information communication terminal apparatus, said glycan profile classification apparatus including a control component and a memory component, said apparatuses being communicatively connected to each other via a network;
(1) wherein the information communication terminal apparatus includes
(1a) a glycan profile data sending unit that transmits the glycan profile data of a cell of interest obtained from a subject to the glycan profile classification apparatus;
(1b) a result-receiving unit that receives the result of the glycan profile classification of the cell of interest transmitted from the glycan profile classification apparatus;
(2) wherein the glycan profile classification apparatus includes
(2a) a glycan profile data-receiving unit that receives glycan profile data derived from the cell of interest obtain from the subject transmitted from the information communication terminal apparatus;
(2b) a data comparison unit which compares the data from the data-receiving unit with the data stored in the memory unit;
(2c) a classifier unit that determines the status of the cell of interest from the subject, based on the results of the data comparison unit; and
(2d) a classification result-sending unit that transmits the classification result of the cell of interest obtained by the classifier unit to the information communication terminal apparatus; and
wherein the memory unit contains glycan profile data for the same cell type as that of the cell of interest.

The glycan profile classification system may be used to determine tumor or non-tumor phenotype of the cell of interest obtained from the subject where the memory unit contains glycan profile data relating to tumor or non-tumor cells.

As a result, the system can compare the glycan profile determined from the cell of interest obtained from a subject with the glycan profiles held in the memory unit that are representative of the cell of interest, e.g. representative of a tumor cell or normal cell, or virus-infected cell, or normal cell. In this way, the cell of interest can be identified as tumor or non-tumor, virus-infected or non-virus-infected. Other status classifiers for cell could also be determined in the same way.

Although the system may be used to perform independent classification of cell status, such as cell phenotypes, i.e. tumor v non-tumor, virus-infected v non-virus infected, recurrence phenotype v non-recurrence phenotype, drug susceptibility profile, and primary tumor v secondary (metastatic tumor), it is preferred that the data contained within the memory unit of the system will allow a sample to be classified as multiple status phenotypes, e.g. tumor, predicted recurrence and drug susceptibility profile.

In one embodiment, the system further comprises the means to add the inputted data via the data sending unit to the stored data already held in the memory unit so that this new data can be included in the analysis performed by the data comparison unit. In this way the glycan profile data representative of a particular cell status (e.g. tumor or non-tumor) is constantly updated.

The status of the cell of interest is selected from the group consisting of aging, cellular activity, multipotency, differentiated directivity and effectiveness.

In a preferred embodiment, the glycan profile data derived from the cell of interest obtained from the subject is obtained by a method of the first aspect of the invention.

In accordance with this further aspect of the invention, there is also provided a glycan profile classification program that makes an information processing apparatus including a control component and a memory component execute a method of determining and/or classifying the glycan profile of a cell of interest obtained from a subject, the method comprising:
(i) a comparing step of comparing data based on the glycan profile of the cell of interest obtained from a subject with the glycan profile data stored in the memory component; and
(ii) a classifying step for classifying the glycan profile data of the cell of interest from said subject, based on the comparison calculated at the comparing step; and wherein said cell is classified into phenotypes including tumor, non-tumor; virus-infected, non-virus-infected, tumor recurrence, tumor non-recurrence; primary tumour, secondary (metastatic tumor) and/or drug susceptibility.

In accordance with this aspect of the invention, there is also provided a computer-readable recording medium, comprising the glycan profile classification program described above recorded thereon.

In respect of all aspects of the invention described herein, the cell from which the glycan profile data is derived may be obtained from a subject already diagnosed with a disease such as cancer or viral infection or it may be obtained from a subject suspected of having a disease. Accordingly, with regard to the latter, the classification of the glycan profile may also include the diagnosis.

Aspects and embodiments of the present invention will now be illustrated, by way of example, with reference to the accompanying figures. Further aspects and embodiments will be apparent to those skilled in the art. All documents mention in this text are incorporated herein by reference.

### BRIEF DESCRIPTION OF DRAWINGS

FIG. 1: Comparison chart for glycan profiling.

FIG. 2: Drawing explaining the flow of deep learning used in the invention.

### DETAILED DESCRIPTION

The present invention provides a method for determining a glycan profile of a cell of interest without the destruction of the cells themselves. This provides an improvement over the methods described in the art.

Further disclosed herein is a glycan analyser which detects a fluorescently labelled complex of a glycan and an immobilised glycan binding protein on a solid support and measures the level of fluorescent intensity generated following the excitation light applied to the immobilised complex. The glycan analyser may comprise one or more or any combination of the following features.

(i) means for detecting fluorescence and measuring its intensity;
(ii) means for storing the measured intensity levels;
(iii) means for comparing the measured intensity levels with stored intensity levels obtained from known labelled glycan-glycan binding protein complex;
(iii) means for providing an output relating to the comparison of the measured intensity levels and the stored intensity levels.

The glycan analyser of the invention may comprise a database for classifying the fluorescent intensity levels for a plurality of glycan-glycan binding protein complexes where the glycan is of known structure. This forms a memory means.

The glycan analyser of the invention may further comprise computer means for calculating the fluorescent intensity for the glycans under test and comparing these to known values. The computer means may then provide an output via a display screen showing the results of the comparison.

The term glycan as used herein refers to the carbohydrate portion of a glycoconjugate, such as a glycoprotein, glycolipid, or a proteoglycan, even if the carbohydrate is only an oligosaccharide. Glycans usually consist solely/either or both of N- and O-glycosidic linkages of monosaccharides. Glycans can be homo- or heteropolymers of monosaccharide residues, and can be linear or branched. There is no restriction to the glycan that can be identified using the method of the invention. Common glycans include glycoside protein type glycan (N-binding type glycan and O-binding type glycan), glycolipid type glycan, glycosamino glycane type glycan, and polysaccharide-derived oligo saccharide chain. Glycan binding proteins can be any protein or peptide capable of binding to a glycan (sugar chain), for example, lectins, enzymes with a sugar-binding domain, cytokines having binding affinity for sugar chain molecules or antibody binding domains capable of binding to sugar chains. In a preferred embodiment, the glycan binding protein is a lectin.

Lectins are a well-known class of proteins belonging to various molecule families which can be obtained from animals and plants, fungus, bacteria, and viruses. For example, lysine B chain-related “R type lectin” is found in bacteria, “calnexin, calreticulin” found generally in eukaryotes and concerning folding of glycoside protein, calcium demanding “C-type lectin” found in multicellular animals and containing may typical lectins, “galectin” found in animals and showing specificity to galactose, “leguminous lectin” found in plants, and “L-lectin” having structural similarity therewith and concerning animal intracellular transportation, man nose 6-phosphate binding “P-lectin” concerning intracellular transportation of lysosomal enzyme, “annexin” binding to acidic glycan including glycosamino glycan, and “I-type lectin” belonging to immunoglobulin superfamily and including “siglec”. Other lectins will be known to the skilled person. The lectins are immobilised to the solid support and therefore may be adapted to facilitate this. For example, the lectin may comprise a tag (e.g. additional amino acid sequence) or may contain site specific mutations, or even chemical modification.

The solid support may be any substrate capable of photoconductivity and capable of generating evanescent waves on the surface by the excitement light. Examples of such substrates include glass, quartz glass and synthetic quartz glass. In a preferred embodiment, the substrate is glass. The lectin may be immobilised on the substrate via a substance coated onto the substrate. Commonly this coating comprises a compound having an epoxy group as an active group. Such compounds include 3-glysidoxy propyl trimethoxy silane (GTMS), 2-(3,4 epoxy cyclohexyl) ethyl trimethoxy silane, 3-glysidoxy propyl methyl diethoxy silane, 3-glysidoxy propyl triethoxy silane, or a silane coupling compound having a plurality of epoxy groups at the top ends of branched spacers. Alternatively other coupling compounds containing polyethylene glycol, protein, biotin-avidin may be used.

It is preferable to have a plurality of lectins immobilised on the solid support in the form of a microarray. Lectin microarrays are commercially available, for example, LecChip™; GlycoTechnica. Lectins can be spotted onto the coated solid support and immobilized thereon by interaction with the coating material, e.g. by co-valent bonding via amino groups. The number of glycan binding proteins on the microarray can vary from 10 to 100, preferably between 20 and 80, more preferably between 30 and 60. Commercially available evanescent-field fluorescent-assisted (EFF) lectin array system commonly use a glass slide spotted with 45 lectins.

Fluorescence intensity may be measured by a suitable scanner such as commercial available scanners, e.g. GlycoStation™; Reader 1200 (GlycoTechnica), and the data analysed using GlycoStation® Tools Pro Suite 1.5 (GlycoTechnica).

The cells of interest may be cultured by standard methods and protocols known in the art. It is particularly preferred that the culture medium is serum-free.

Analysis method of the invention is described in detail below.

Method of testing and analysis in this invention analyzes characteristics attributed to cells in a non-destructive manner by utilizing glycan profiling using glycan proteins' lectin microarrays secreted in the cell culture medium.

“Analyzing characteristics attributed to cells in a non-destructive manner” means analyzing cell types or cell conditions without destroying cells.

According to the invention's method of testing and analysis, it will be possible to analyze characteristics of the cells at low cost, not by using cells to be inspected but by using culture mediums which are to be discarded through such processes as medium exchange. With the method of testing and analysis of the invention, cell conditions are analyzed in a non-destructive manner by obtaining glycan profile of the cells from glycoconjugates secreted in the culture mediums, using microarrays with glycan binding proteins immobilized on the surface.

Regarding the analysis method in the invention, process of obtaining glycan profile is enabled by using evanescent wave excitation fluorescence scanner.

Evanescent wave excitation fluorescence scanner is a device which enables detection of interaction between glycoconjugates, secreted in the aforementioned culture mediums of the above cells, which is the object of the analysis, and the aforementioned glycan binding proteins, by generating evanescent wave (near field) on the surface of microarrays which has glycan binding proteins, e.g. lectins, immobilized on it, without washing or drying the surface of the above mentioned microarray, in a non-destructive manner. Further as the glycoconjugates are obtained from the supernatant of the cell culture medium into which they have secreted, there is no need for centrifugation which destroys the cells and requires removal of cell debris.

Examples of evanescent wave excitation fluorescence scanner include GlycoStation Reader and GlycoLite manufactured by GlycoTechnica Ltd.

Cells to be tested and analyzed with the invention is not limited to a specific kind of cell as long as the invention can be employed effectively. Human cell is an example of such a cell. Cell types to be tested and analyzed in this invention is not limited to a specific cell type, as long as the invention proves to be effective. Pluripotent cells such as differentiated cells and MSCs are examples of such cell types.

State of the cells to be tested and analyzed is not limited to a specific cell condition, as long as the invention can be employed effectively. Aging and activity, multipotency, differentiated directivity and effectiveness are examples of such cell conditions.

Culture medium to be used in the invention is not restricted to a particular type as long as the medium can cultivate cells. Serum free culture medium is a preferred example. Glycoconjugate to be used in this invention is not restricted to a particular type as long as the invention can be employed effectively. Glycoprotein is an example of such a glycoconjugate.

Serum free culture medium is a medium for culturing animal cells which does not contain serum. In contrast with culture medium added with approximately 5-20% serum in order to supplement necessary components such as growth factor when artificially cultivating animal cells, it achieves the same effect without the addition of serum.

With the invention, it is possible to use machine learning, in particular, supervised learning. ‘Supervised learning’ is a learning method where a unique procedure of achieving the result is found by learning repeatedly from data and finding hidden patterns. This does not rely on hard coding the procedure in the software to derive the intended result. With this invention, it is possible to use deep learning as machine learning for the analysis method. ‘Deep learning’ is a method of supervised learning which analyses through learning the data by repeating several layers of neural networks which reference information transmission between nerve cells.

For the method of testing and analysis in the invention, evanescent wave fluorescent excitation light scanner, which enables detection of glycans with weak interactions without omission, sample chip analysis device and evanescent wave excitation fluorescence scanner for obtaining glycan profile can be used.

For the method of testing and analysis in this invention, the method to analyze interaction between protein and glycan, which is described in patent literature 1, 2 and 3, can be utilised. These documents are incorporated herein by reference. For the method of testing and analysis in this invention, the device for testing and analyzing glycan or complex carbohydrate, as described in patent literature 1, 2 and 3, can be utilised. For the method of testing and analysis in this invention, it is possible to use other existing technology.

Examples of application of the invention is described below. The invention is not restricted to the application formats as stated above and to the application examples stated below. It can also be changed in various ways within the technological scope which can be understood from the descriptions listed in the claims of the invention.

### Examples

Human adipose-derived MSCs (hereinafter referred to as ‘AD’), human umbilical cord-derived MSCs (hereinafter referred to as ‘UC’), human lung-derived fibroblasts (hereinafter referred to as ‘Lung’), human liver-derived fibroblasts (hereinafter referred to as ‘Liver’), human artery-derived cells (hereinafter referred to as ‘Aorta’), were cultured in a serum-free medium for MSCs (developed by Rohto Pharmaceuticals Co. Ltd.).

20 μL of culture supernatant was collected, after the 2nd and the 4th day of cultivation, and the samples were pretreated according to the protocol listed below.

1. 20 μL of each of the samples and Cy3 Mono-Reactive dye 100 μg labelling (GE Healthcare, PA23001 subdivided into 100 μg labelling) were mixed and left to react for 1 hour at room temperature in a dark room.

2. Desalting column (Zeba (trademark)) Spin Desalting Columns, 7K MWCO (Thermo SCIENTIFIC, 89882) was centrifuged at 1,500×g for 1 minute at 4° C.

3. TBS=300 μL was applied to desalting column and was centrifuged at 1,500×g for 1 minute at 4° C. (column cleaning). This process was repeated twice.

4. Full quantity of each of the samples and T=25 μL were applied to the desalting column and was centrifuged at 1,500×g for 2 minutes at 4° C. and the unreacted Cy3 was removed.

5. Each sample was applied with 450 μL of probing solution and was diluted in measuring flasks to 500 μL/tube.

6. After washing LecChip (registered trademark) (lectin microarrays manufactured by GlycoTechnica) 3 times with probing solution (100 μL per well) (probing solution for LecChip manufactured by GlycoTechnica), each sample, Cy3-labeled fraction, (100 μL per well) was applied and LechChip (registered trademark) was made to react for over 17 hours at 20° C.

7. The measurement was performed on LecChip (trademark) in a liquid phase wherein the applied Cy3-labeled fraction was still reacted with GlycoStation (trademark) Reader, an evanescent wave fluorescent excitation light scanner manufactured by GlycoTechnica (measurement conditions: cumulative number: 4 times, exposure time: 133 msec, camera gain: 75, 85, 95, 105, 115, 125).

8. Digitization and data integration (gain integration) was conducted using GlycoStation (trademark) Tools Pro Suite 1.5 (glycan analysis software manufactured by GlycoTechnica).

9. All the digitized data, which have been data integrated (gain integrated), were exported to Microsoft (trademark) Excel sheet. This data can be exported to other spreadsheet software.

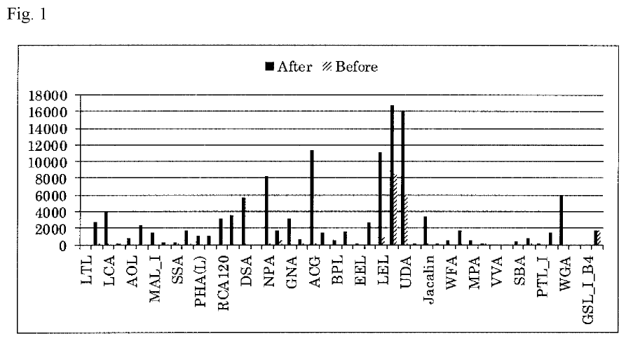

Glycan profiling was conducted on samples, Cy3-labeled fractions which were pretreated as above, based on the strength of fluorescence (glycan profile) obtained by scanning 45 lectins, immobilized on lectin microarrays, (LecChip (trademark)) using glycan profiler (GlycoStation (trademark) Reader 1200). Comparison chart of the result is depicted in FIG. 1, which represents glycan profiling in the culture medium ‘Before’ and 2 days ‘After’ cell culture.

No signals other than STL and UDA were detected from supernatant of culture medium before cell culture and numerous lectin signals were detected from supernatant of culture medium after 2 days cell culture. Though not shown in the diagram, an increased amount of lectin signals were detected from supernatant of medium after 4 days cell culture compared with that of supernatant of medium after 2 days cell culture. This confirmed that numerous signals from lectins seen after cell culture were function factors secreted from cells within the culture medium.

Featured vector, to be used for both supervised and unsupervised learning was derived from the strength of fluorescence (glycan profile) obtained by scanning a lectin microarray (LecChip (trademark)) containing 45 distinct lectins, as indicated in FIG. 1, using glycan profiler (GlycoStation (trademark) Reader 1200).

Normalization was carried out to acquire common logarithms by adding 1.0 to the strength of fluorescence, so to emphasize small differences and reduce large differences in the strength of fluorescence in order for the minimum value of the common logarithms to be 0 and the maximum value to be 1.

Normalization method (x: measured strength of fluorescence, xₘᵢₙ: minimum strength of measured fluorescence, xₘₐₓ: maximum strength of measured fluorescence, y: after normalization)

y=(log(x+1.0)−log(xₘᵢₙ+1.0))/(log(xₘₐₓ+1.0)−log(xₘᵢₙ+1.0))

Supervised learning was performed using linear classification, using multiple classification function classifier included in Jubatus (http://jubat.us/), online machine learning distributed processing framework. And deep learning was performed using TensorFlow (http://tensorflow.org/) and its interface Keras (http://keras.io/), a framework compatible with deep learning,

Because the number of samples was not large, optimisation of hyperparameter for these was done with leave-one-out cross-validation. In order to improve the eventual recognition accuracy, configuration changes were repeated, outputting the same number of recognition accuracy of the classification apparatus as that of the sample.

Though it is not possible to expect similar level of recognition accuracy as with deep learning, linear classification was utilised since it is possible to find out how much influence, strength of fluorescence for each lectin had on the classification. Selection of algorithms such as AROW, were used in classifier in order to maximise recognition accuracy. Furthermore, optimisation of regularization weight value etc., configurable with such algorithms was done. From the completed linear classification apparatus, ranking for lectins which contributed to each classification was produced. This was used to support the results obtained from deep learning, which is described later.

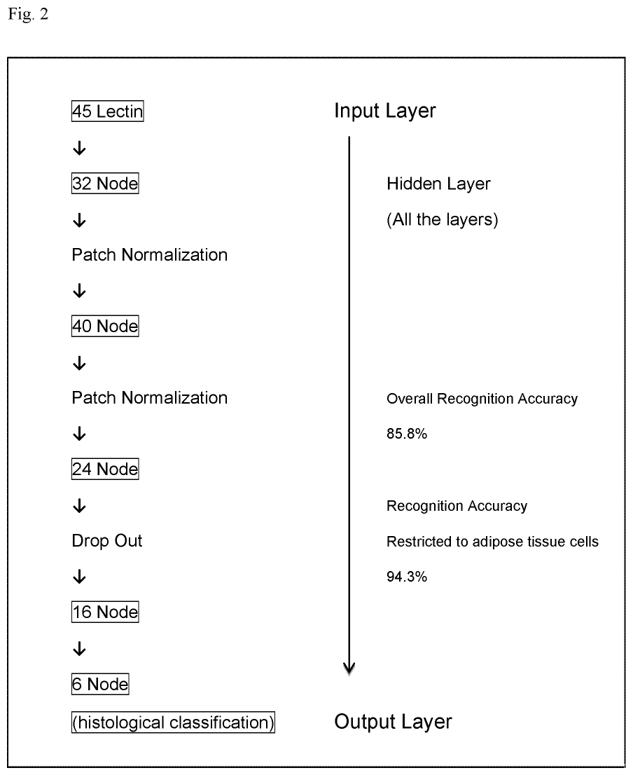

Deep learning was configured by accumulating hidden layers resulting from affine layers (all the binding layers). ReLU function was used for active function and initial value for He was used as initial value for weight. In the case of binary classifications, singular node Sigmoid function was used for the output for the output layer and binary classification is made possible depending on whether it is close to 0 or 1. In the case of multiclass classifications, classification becomes possible by adjusting the node value of the output layer to the number of classification. The following was considered for optimisation of hyperparameter: number of hidden layers to be approximately 2 to 7; node value for each layer to be approximately 4 to 60; batch size; use of batch normalization; drop out value; learning rate; consideration of parameter renewal method other than SGD. Specifically, deep learning was performed with the configuration where an input layer resulted in an output layer as indicated in FIG. 2.

Recognition accuracy for cell types according to deep learning was as described in Table 1. Number of samples used were: AD=105, UC=30, Lung=6, Liver=6, Aorta=6

TABLE 1
Total accuracy: 85.5%

| | |
|---|---|
| AD recognition accuracy | 94.3% |
| UD recognition accuracy | 90.0% |
| Lung recognition accuracy | 33.3% |
| Liver recognition accuracy | 33.3% |
| Aorta recognition accuracy | 16.7% |

Table 1 indicates that MSCs (AD, UD) were recognised at an accuracy rate of more than 90%, which is a significant performance in terms of evaluating cell types, which is the basis for evaluating quality verification. The outcome emphasizes the fact that the result for the cells, which were the subject of the test, was obtained in a non-destructive manner from culture mediums, which would, under a normal circumstance, be discarded, without any special pretreatment.

The invention provides a non-destructive method of testing and analysing, amongst other things, cell types such as pluripotent cells, including differentiated cells and MSCs. The invention also provides a non-destructive method to test and analyse cell conditions, such as aging, activity, multipotency, differentiated directivity and effectiveness.

The description above describes materials and methods of the present invention. The invention may be modified in ways which will be apparent to the skilled person. Accordingly, it is not intended that the invention is limited to the specific embodiments disclosed herein, but that it covers all modifications, adaptions and alternations within the true spirit and scope of the invention.

All references cited herein, including but not limited to published and unpublished applications, patents, and literature references, are incorporated herein by reference in their entirety and are hereby made a part of this specification.

1. A method for determining the glycan profile of a cell of interest, said method comprising the steps of
(a) culturing the cell of interest in a culture medium for a period of time;
(b) following said period of time, collecting a sample of supernatant from the culture medium, wherein the supernatant is cell-free;
(c) mixing the cell-free supernatant with a fluorescent labelling agent to create a labelled sample solution, wherein the fluorescent labelling agent is capable of fluorescently labelling a glycoconjugate;
(d) contacting the labelled sample solution with a plurality of glycan binding proteins under conditions suitable to allow binding of a fluorescently labelled glycoconjugate to bind to at least one of said plurality of glycan binding proteins thereby forming a fluorescent labelled glycoconjugate-glycan binding protein complex, wherein said plurality of glycan binding proteins are immobilized on a solid support;
(e) applying an excitation light and measuring the level of intensity of excited fluorescence generated from the fluorescent labelled glycoconjugate-glycan binding protein complex;
(f) determining a glycan profile of the cell of interest based on the intensity of excited fluorescence generated.

2. The method of claim 1 wherein the glycoconjugate is a glycoprotein.

3. The method of claim 1 wherein the cell of interest is a human cell.

4. The method of claim 1 wherein the cell of interest is selected from a stem cell, a pluripotent stem cell, or a differentiated cell.

5. The method of claim 1 wherein the cell of interest is a human mesenchymal stromal cell.

6. The method according to claim 1 wherein the culture medium is a serum-free culture medium.

7. The method of claim 1 wherein the period of time is greater than 48 hours.

8. The method of claim 1 wherein the fluorescent labelling agent is selected from the group consisting of 2-aminopyridine, Cy3, Cy3.5, Cy5 and tetramethyl rhodamine.

9. The method of claim 8 wherein the fluorescent labelling agent is Cy3.

10. The method of claim 1 wherein the glycan binding protein is selected from lectins, enzymes with a sugar-binding domain, cytokines having binding affinity for sugar chain molecules or antibody binding domains capable of binding to sugar chains.

11. The method of claim 10 wherein the glycan binding protein is a lectin.

12. The method of claim 1 wherein the excitation light is an evanescent wave.

13. The method of claim 12 wherein step (e) is performed by an evanescent wave excitation fluorescence scanner.

14. A method of diagnosing, assessing,
and/or prognosing, a disease in a subject, the method comprising:
(i) comparing a glycan profile for a cell of interest obtained from the subject with a reference glycan profile for the same cell type, wherein said glycan profile for the cell of interest has been determined by a method according to claim 1; and
(ii) determining any differences in the glycan profile between the cell of interest obtained from the subject and that of the reference cell, wherein any difference between the glycan profile of the cell of interest and that of the reference cell is indicative of the presence, absence or degree of disease in a subject.

15. A method according to claim 14 wherein the disease is selected from the group consisting of cancer, virus infection, bacterial infection and autoimmune disease.

16. A glycan profile classification system comprising a glycan profile apparatus and an information communication terminal apparatus, said glycan profile classification apparatus including a control component and a memory component, said apparatuses being communicatively connected to each other via a network;
(1) wherein the information communication terminal apparatus includes
(1a) a glycan profile data sending unit that transmits the glycan profile data of a cell of interest obtained from a subject to the glycan profile classification apparatus;
(1b) a result-receiving unit that receives the result of the glycan profile classification of the cell of interest transmitted from the glycan profile classification apparatus;
(2) wherein the glycan profile classification apparatus includes
(2a) a glycan profile data-receiving unit that receives glycan profile data derived from the cell of interest obtain from the subject transmitted from the information communication terminal apparatus;
(2b) a data comparison unit which compares the data from the data-receiving unit with the data stored in the memory unit;
(2c) a classifier unit that determines the status of the cell of interest from the subject, based on the results of the data comparison unit; and
(2d) a classification result-sending unit that transmits the classification result of the cell of interest obtained by the classifier unit to the information communication terminal apparatus; and
wherein the memory unit contains glycan profile for the same cell type as that of the cell of interest.

17. A glycan profile classification system according to claim 16 wherein status of the cell of interest is selected from the group consisting of aging, cellular activity, multipotency, differentiated directivity and effectiveness.

18. A glycan profile classification system according to claim 16 or claim 17 wherein the glycan profile data derived from the cell of interest obtained from the subject is obtained by
(a) culturing the cell of interest in a culture medium for a period of time;
(b) following said period of time, collecting a sample of supernatant from the culture medium, wherein the supernatant is cell-free;
(c) mixing the cell-free supernatant with a fluorescent labelling agent to create a labelled sample solution, wherein the fluorescent labelling agent is capable of fluorescently labelling a glycoconjugate;
(d) contacting the labelled sample solution with a plurality of glycan binding proteins under conditions suitable to allow binding of a fluorescently labelled glycoconjugate to bind to at least one of said plurality of glycan binding proteins thereby forming a fluorescent labelled glycoconjugate-glycan binding protein complex, wherein said plurality of glycan binding proteins are immobilized on a solid support;
(e) applying an excitation light and measuring the level of intensity of excited fluorescence generated from the fluorescent labelled glycoconjugate-glycan binding protein complex; and
(f) determining a glycan profile of the cell of interest based on the intensity of excited fluorescence generated.

19. A glycan profile classification program that makes an information processing apparatus including a control component and a memory component execute a method of determining and/or classifying the glycan profile of a cell of interest obtained from a subject, the method comprising:
(i) a comparing step of comparing data based on the glycan profile of the cell of interest obtained from a subject with the glycan profile data stored in the memory component; and
(ii) a classifying step for classifying the glycan profile data of the cell of interest from said subject, based on the comparison calculated at the comparing step; and wherein said cell is classified into phenotypes including tumor, non-tumor; virus-infected, non-virus-infected, tumor recurrence, tumor non-recurrence; primary tumour, secondary (metastatic tumor) and/or drug susceptibility.

20. A computer-readable recording medium, comprising the glycan profile classification program of claim 19 recorded thereon.
